(19) United States
(12) Patent Application Publication
(10) Pub. No.: US 2010/0227410 A1
(43) Pub. Date: Sep. 9, 2010

(54) MULTI-IMMUNOAFFINITY BASED ANTIGEN IDENTIFICATION

(75) Inventors: Laszlo Takacs, Newbury Park, CA (US); Janos Kadas, Debrecen (HU); Andras Guttman, San Diego, CA (US)

Correspondence Address: NIXON & VANDERHYE, PC, 901 NORTH GLEBE ROAD, 11TH FLOOR, ARLINGTON, VA, 22203, US

(73) Assignee: BIOSYSTEMS INTERNATIONAL SAS

(21) Appl. No.: 12/451,180

(22) PCT Filed: May 5, 2008
(86) PCT No.: PCT/EP2008/055492

§ 371 Date: May 25, 2010

Related U.S. Application Data
(60) Provisional application No. 60/915,941, filed on May 4, 2007.

(51) Int. Cl. G01N 33/00; C07K 1/22; G01N 33/566; G01N 33/559; G01N 33/50

(52) U.S. Cl. 436/86; 530/413; 436/501; 204/461; 204/450

(57) ABSTRACT

The present invention relates to methods for identifying antigens from complex analyte samples. More specifically, the invention relates to methods for identifying antigens recognized by monoclonal antibodies in a complex analyte sample. The methods of this invention use a first affinity chromatography via a “multiaffinity” step and a second, “singleaffinity” step that implements parallel affinity chromatography.

The present invention relates to methods for identifying antigens from (complex) analyte samples. More specifically, the invention relates to methods for identifying antigens recognized by monoclonal antibodies in a (complex) analyte sample.

### GENERAL INTRODUCTION

Generation of bioactive solid surfaces through immobilization of antibodies is important for biomarker discovery and screening. The solid-phase environment provides sufficient bioactivity, stability and reproducibility without a high background (nonspecific binding) or loss of antigens. The progress in microfabrication technologies and the trend towards the creation of integrated biodevices imposes a new and major constrain on immobilization techniques, for example, the requirement for highly defined space-programming of the immobilization of biomolecules. These will be used in high-throughput screening (HTS) processes such as for protein ID of cognate antigens of mAbs from libraries specific for individual elements of complex analytes like the normal human plasma or plasma from diseased subject.

The problem of high throughput cognate antigen protein IDs of mAbs from libraries of mAbs, such as libraries specific for the normal or disease human plasma is that the majority of proteins represent no interest, yet these proteins contaminate the purified analyte of interest and therefore reduce the success rate of the MS based or other identification processes.

Here, we present a novel method that allows the identification of desired antigens from complex analyte samples. The invention more specifically provides novels methods for treating complex samples, that permit antigen isolation and/or identification. The methods of this invention use a two step procedure; first affinity chromatography via a “multiaffinity” step that contains minimum one or a set of mAbs as capture reagents, then, second, “singleaffinity” step that implements parallel affinity chromatography. The product of the first step is loaded onto the second step. Comparative analysis of composition of the flow through and eluates from the second step process allows identification of analyte species that represent specifically depleted and corresponding (e.g. shared physicochemical characteristics such as molecular mass) specifically enriched components that are unique to a given mAb, present as a capture reagent on the second affinity chromatography step. Physicochemical characteristics based separation method such as SDS gel-electrophoresis is used then to further purify the mAb specific components which are then submitted for mass spectrometry based protein identification.

Both the “fullscale” and the “miniaturized” nanoscale and microfluidics based versions of the process are subject of the present patent application.

### SUMMARY OF THE INVENTION

The present invention relates to methods for identifying and/or isolating proteins. The invention particularly relates to methods of identifying antigens that are bound by antibodies of interest. The invention resides, in particular, in a process which comprises subjecting a sample to be analysed to a first multiaffinity binding step and, then, to at least one singlyaffinity binding step. The method is particularly advantageous in identifying, from complex samples, antigens recognized by antibodies of interest.

### BRIEF LEGEND TO THE FIGURES

FIG. 1: Steps of the sequential identification of each individual fragment from a digested affinity purified analyte species. The identification of the mass of the fragments permits high fidelity protein ID assignment with the use of current empiric and predicted protein data sets.

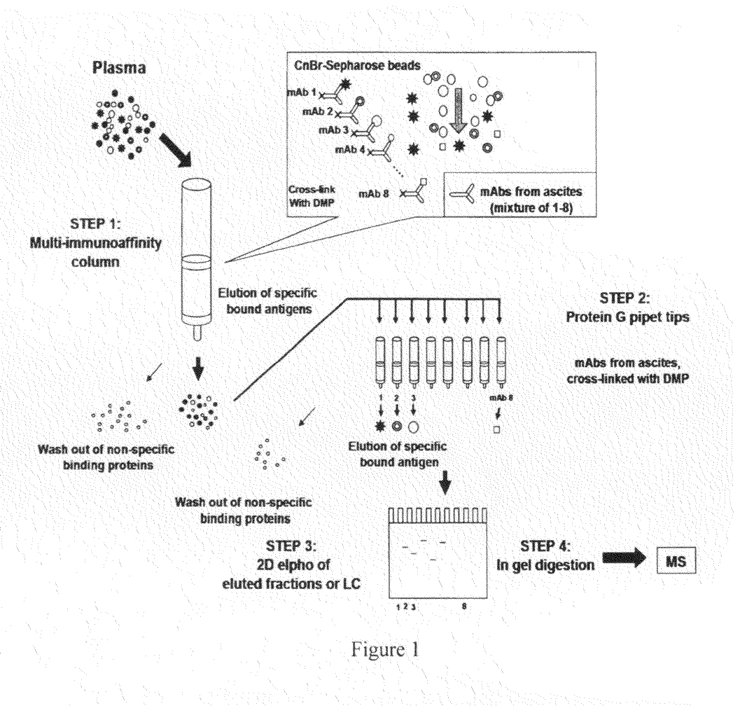

FIG. 2. Composition analysis via SDS gel electrophoresis of multiaffinity column eluate (Step 1) and single affinity column array flow through and eluates (Step 2). Bands shown in boxes are specific for individual mAbs (listed 1-8).

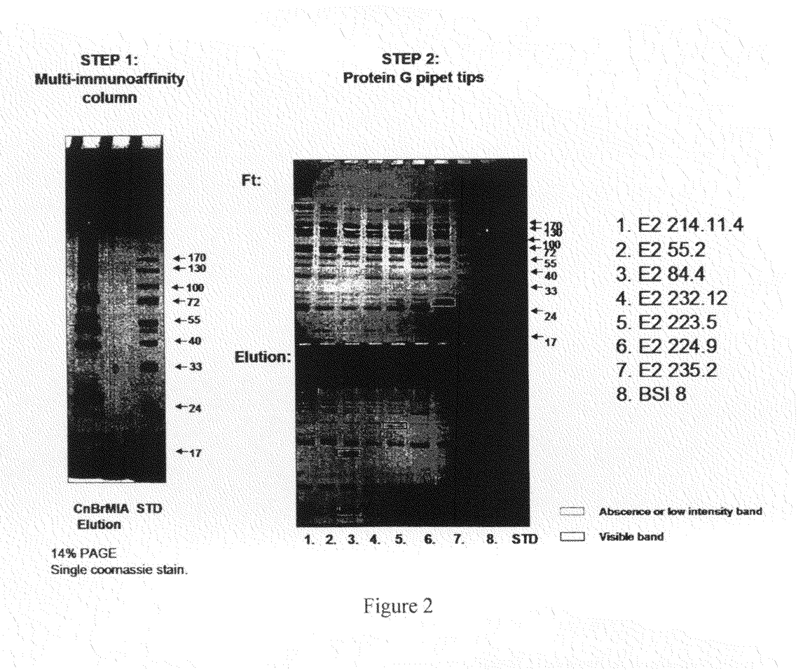

### DETAILED DESCRIPTION OF THE INVENTION

The invention relates to a method of protein isolation and/or identification comprising the steps of:
contacting a sample containing proteins (preferably at least part of said proteins being unknown or uncharacterized) with a multi-affinity support comprising a plurality (e.g., from 3 to 50, from 5 to 40, from 5 to 30, from 5 to 20) of binding reagents;
recovering the material bound to said multi-affinity support;
separately contacting said material with a plurality of single-affinity supports, each of said single-affinity supports comprising a single binding reagent as contained in the multi-affinity support; and
isolating and/or identifying a protein that binds to a binding reagent in at least one of said single-affinity supports.

Preferably, the sample is a complex sample comprising a plurality of proteins, such as a biological fluid, e.g., plasma, blood, serum, bronchioalveolar fluid, urine, sputum, exudates, particularly of human origin. The sample may also be selected from human biopsy material, human tissue section, human feces, etc., more generally from any material comprising a mixture of proteins. The sample may be pre-treated, e.g., to normalize or reduce the complexity of the sample components.

Preferably, the binding reagents are specific for components of a complex biological fluid, such as human plasma. The binding reagents are preferably monoclonal antibodies, or derivatives thereof (e.g., Fab fragments, ScFv, etc.) having essentially the same antigen specificity.

The supports may be any partitioning supports. In a particular embodiment, the supports are chromatography supports, more preferably columns. Other supports may be beads, such as magnetic beads, possibly in pipette tips.

In a specific embodiment, the sample is human plasma or serum, and the binding reagents are monoclonal antibodies.

The invention is particularly suited for isolating and/or identifying antigens that are bound by monoclonal antibodies.

The first multiaffinity step is preferably performed using a multiaffinity support prepared as follows. One or, most preferably, a plurality of distinct antibodies (e.g., from 5 to 20) are immobilized on a suitable affinity support. The antibodies are typically immobilized through their Fc portion, more preferably by covalent linkage or crosslinking. The antibodies may be monoclonals, e.g., purified or in the form of hybridoma supernatant, ascites, serum or fermentation fluid. In a preferred embodiment, a plurality of, typically 5-20 different purified mAbs or mAbs from hybridoma supernatant are loaded and crosslinked via their Fc portion onto an appropriate affinity chromatography column (e.g., HiTrap Protein G, Protein -L or CNBr activated Sepharose) allowing the rest of the hybridoma supernatants, or non bound mAbs to exit the system (Multi-Immuno-Affinity (MIA) column preparation).

To perform the multiaffinity step, the appropriate amount of sample (antigen-containing media, such as human plasma) is loaded on the support. In a preferred embodiment, the sample is either pre-treated and/or flown through a mock irrelevant Ab containing column, to remove high abundant proteins prior to be loaded onto the MIA column. This step supports affinity binding of the corresponding antigens and decreased nonspecific interactions.

The affinity support or column is then washed extensively to remove any remaining non-specifically bound species, and the bound antigens are eluted from the mAb(s) covered affinity surface.

The second step of specific purification of individual antigens by single affinity (microcolumn) array technology is typically performed as follows:

Each individual mAb is load and crosslinked via their Fc portion onto an affinity microcolumn array (e.g., Protein G, Protein L pipet tips), allowing the rest of the hybridoma supernatants to exit the system. An appropriate portion of the product obtained from step 1 is then loaded onto the individual columns/tips of the microcolumn array. This step supports affinity binding of corresponding antigen only in a low microliter scale. In a particular embodiment, the product of step 1) is subjected to an ELISA or dot-blot assay as disclosed in WO/2006/043179, prior to step 2. The bound antigens are then eluted from the individual affinity microcolumn array columns.

In a preferred embodiment, the protein identification step comprises subjecting an eluate of at least one of said single-affinity chromatography supports to an electrophoresis, and isolating a protein band of interest. Most preferably, both the eluate and flow through of said at least one single-affinity chromatography support are subjected to electrophoresis, and a protein band that is specific or amplified in the eluate as compared to the flow through is isolated.

Preferred specific steps for said identification step are disclosed below:
1. Slab gel electrophoresis of the individual flow through and eluate samples from the affinity microcolumn array;
2. Comparative analysis for the identification of mAb specific depleted (flow through) and corresponding enriched (eluate) composition (e.g. stained gel images);
3. After visualization, cut off bands of interest;
4. In gel digestion of cutoff bands, sample preparation, digestion and nano LC-MS analysis to provide protein ID.

The invention may be used, e.g., for identifying antigens characteristic of a disease or trait in a mammal, preferably a human. In this regard, a particular object of this invention resides in a method for isolating and/or identifying antigens specific for a disease or trait of a mammal, comprising the steps of (i) producing antibodies specific for components of a fluid from said mammal and (ii) identifying antigens recognized by said antibodies using a method as described above.

As disclosed in the experimental section, the invention allows the rapid and reliable determination and characterization of antigens recognized by antibodies generated against human plasma. In this regard, the invention has been used to determine the antigen bound by several monoclonal antibodies of interest and the results are listed in Tables 1 and 2, showing the effectiveness of the claimed method.

The invention further relates to the use of monoclonal antibody E2 214.11.4, or a derivative thereof (e.g., Fab fragment, CDR region, ScFv, etc.) for detecting the presence or amount of Complement C4-A precursor in a sample, particularly a biological fluid.

The invention also encompasses the use of monoclonal antibody E2 84.4, or a derivative thereof (e.g., Fab fragment, CDR region, ScFv, etc.) for detecting the presence or amount of Haptoglobin-related protein/Haptoglobin in a sample, particularly a biological fluid.

The invention further relates to the use of monoclonal antibody E2 223.5, or a derivative thereof (e.g., Fab fragment, CDR region, ScFv, etc.) for detecting the presence or amount of C4b-binding protein alpha chain precursor in a sample, particularly a biological fluid.

The invention also resides in the use of monoclonal antibody E2 235.2, or a derivative thereof (e.g., Fab fragment, CDR region, ScFv, etc.) for detecting the presence or amount of Complement Clq subcomponent subunit B precursor in a sample, particularly a biological fluid. The invention also relates to a binding reagent selected from monoclonal antibodies E2 214.11.4, E2 84.4, E2 223.5 and E2 235.2, or derivatives thereof (e.g., Fab fragment, CDR region, ScFv, etc.) having essentially the same antigen specificity.

Further aspects and advantages of this invention will be disclosed in the following examples, which shall be considered as illustrative only and do not limit the scope of the present application.

### EXAMPLES

### Example-1

### Multi Immunoaffinity Column Chromatography

Bead: CNBr-activated Sepharose 4B (Coupling capacity: 25-60 mg α-chymotrypsinogen/ml drained medium pH stability: 2-11)

1. Media preparation: 1 g lyophilized powder gives about 3.5 ml final volume of medium, and 5-10 mg protein ligand per ml medium is recommended.
Weigh out the required amount of powder, and suspend it in 1 mM HCl
   2 g powder, gives 7 ml final volume of medium
Wash the medium washed for 15 minutes with 1 mM HCl on a sintered glass filter
   Approximately 200 ml 1 mM HCl per gram freeze-dried powder needed, in several aliquots.

### 2. Coupling and Blocking

Dissolve the ligand to be coupled in coupling buffer
   Buffer: 0.1 M NaHCO3 pH 8.3, 0.5 M NaCl
   About 5 ml coupling solution/g lyophilized powder is recommended.
   5-10 mg protein ligand per ml medium is recommended.
   Ligand: 2-2 mg purified IgG from ascites (aliquoted in PBS, 10% glycerol)
   (E2 214.11.4; E2 55.2; E2 84.4; E2 232.12; E2 223.5; E2 224.9; E2 235.2; BSI 8)
Add the coupling solution containing the ligand with the prepared medium suspension in a stoppered vessel.
Rotate the mixture end-over-end overnight at 4° C.
   Other gentle stirring methods may be employed.
Wash away excess ligand with at least 5 medium (gel) volumes of coupling buffer 35 ml all Buffer: 0.1 M NaHCO3 pH 8.3, 0.5 M NaCl
Block any remaining active groups. Transfer the medium to a quenching buffer. Let it stand for 15 minutes at 4° C. Buffer: 1 M ethanolamine, pH 8.0.
Wash the medium with at least three cycles of alternating pH. Wash with at least 5 medium volumes of each buffer.
   Buffer I: 0.1 M sodium acetate, pH 4.0 containing 0.5 M NaCl
   Buffer II: 0.1 M Tris-HCl, pH 8.0 containing 0.5 M NaCl

### 3. Antigen Binding

This step should preferably be performed in the cold room.
Sample pH should be the same as that of the binding buffer.

Mix the plasma sample with 2× phosphate binding buffer.
Binding Buffer: PBS 0.1% Triton X-100, 0.05% Sodium Azide
Check the final volume after mixing!
Plasma volume before mixing: 150 ml.
Working mix: 300 ml
Filter the sample through a 0.22 μm or 0.45 μm filter. (or also centrifuge the plasma at maximal speed in 50 ml tubes in the Beckman centrifuge for 30 minutes (6000 rpm)) It can help us to prolong the working life of the medium.
Wash the IgG ligand-medium suspension 2 medium volumes of Binding buffer before mix
Add the plasma sample to the prepared IgG ligand-medium suspension in a stoppered vessel. At once the mix the whole and rotate!!
Incubate and rotate the mixture for 1 day at 4° C.
Before packing remove the plasma sample from medium, and wash several times (5×) with Binding buffer via resuspending the column in 50 ml binding buffer and centrifugation at 400 rpm for 5 minutes in the Beckman centrifuge.

### 4. Packing

Empty column: GE Amersham XK 16/20 (with 1 adaptor, and thermostat jacket) Bed volume: changeable, 5-31 ml; Bed height: changeable, 2.5-15 cm
Prepare slurry with binding buffer; see below, in a ratio of 75% settled medium to 25% buffer. The binding buffer should not contain agents which significantly increase the viscosity.
Package the media after washing steps (see washing step at Binding)
Equilibrate all material to the temperature at which the chromatography will be performed.
De-gas the medium slurry.
Eliminate air from the column dead spaces by flushing the end pieces with buffer. Make sure no air has been trapped under the column net. Close the column outlet with a few centimetres of buffer remaining in the column.
Pour the slurry into the column in one continuous motion. Pouring the slurry down a glass rod held against the wall of the column will minimize the introduction of air bubbles.
Immediately fill the remainder of the column with buffer, mount the column top piece onto the column and connect the column to a pump.
Open the bottom outlet of the column and set the pump to run at the desired flow rate. This should be at least 133% of the flow rate to be used during subsequent chromatographic procedures. If I have packed at the maximum linear flow rate, do not exceed 75% of this in subsequent chromatographic procedures. For the washing step use 0.3 ml/min, and after for the elution increase to 1 ml/min. In this case 1.5 ml/min is used for package.
Maintain the packing flow rate for 3 bed volumes after a constant bed height is reached. Using the adaptor:
After the medium has been packed as described above, close the column outlet and remove the top piece from the column. Carefully fill the rest of the column with buffer to form an upward meniscus at the top.
Insert the adaptor at an angle into the column, ensuring that no air is trapped under the net. Make all tubing connections at this stage. There must be a bubble-free liquid connection between the column and the pump.
Slide the plunger slowly down the column so that the air above the net and in the capillary tubings is displaced by the eluent. Valves on the inlet side of the column should be turned in all directions during this procedure to ensure that air is removed.
Lock the adaptor in position on the medium surface, open the column outlet and start the flow. Pass buffer at packing flow rate until the medium bed is stable. Re-position the adaptor on the medium surface as necessary.

The column is now packed with bound antigens (hopefully) and ready for use.

### 5. Washing

Use a type of detergent to decrease the unspecific binding, like Triton-X100. Make a pH gradient with several step with low salt concentration. Should be performed with cooled buffers and column (7 ml column volume).
Wash the column at least 5 column volumes of Binding buffer, at 0.5 ml/min (1 hour).
Wash the column with 10-12 volumes of Wash Buffer I, at 0.3 mL/min (4-4.5 hour). Buffer: 140 mM NaCl, 50 mM NaH2PO4, pH 6.0, 0.1% TX-100
Wash the column with 3-5 volumes of Wash Buffer II, at 0.3 mL/min (1-1.5 hour). Buffer: 140 mM NaCl, 50 mM NaH2PO4, pH 6.0

### 6. Elution:

Elute the bound fraction with 2 volumes of Elution buffer, at 1 mL/min. Buffer: 0.1 M glycine-HCl, pH 2.0. Fractionation: 1 ml fractions
Neutralize eluted fractions with Neutralization Buffer; Buffer: 1 M Tris-HCl, pH 9.0
   7. QC of eluted fractions

Electrophoresis, in gel digestion, digestion in liquid phase, MS from mixed and concentrated fractions from elution. Optional: Electrophoresis and digestion in liquid phase with MS from separated factions.

8. Regeneration and storage of column
Wash the medium with at least three cycles of alternating pH. Wash with at least 3 medium volumes of each buffer.
   Buffer 1: 0.1 M sodium acetate, pH 4.0 containing 0.5 M NaCl
   Buffer II: 0.1 M Tris-HCl, pH 8.0 containing 0.5 M NaCl
Re-equilibrate the column in Binding buffer
Swollen coupled medium should be stored at 4-8° C. in presence of a bacteriostatic agent.

### Example 2

### Phynexus Chromatography

Bead/column: 1000+PhyTip columns with Protein G resin: Maximum solution volume of 1000 μL, Protein G resin volume 160 μl; Coupling capacity: ˜1000 μg.

System: Computer controlled 8-channel pipet

### 1. Tip Preparing

PhyTip columns with Protein G are stored in Glycerol when shipped from PhyNexus.
Wash the tips with 1 ml PBS
   Program: 900 μl intake/expel
      0.5 ml/min
      Delay (hold): after intake 30 sec, after expel 30 sec
      Cycle: 2×
Repeat this step 2 or 3 times with new PBS solution.

### 2. Coupling and Blocking

Ligand: 0.3 mg purified IgG from ascites (aliquoted in PBS, 10% glycerol).
   1. E2 214.11.4
   2. E2 55.2
   3. E2 84.4
   4. E2 232.12
   5. E2 223.5
   6. E2 224.9
   7. E2 235.2
   8. BSI 8
Pipet the ligand in 200 μl (up with PBS to 200 μl) suspension into a 96-well plate separately, according to the numbers (In case of bigger volume, aliquot the ligand suspension to 200-200 μl and intake it separately).
Intake the antibody solution
   Program: 200 μl intake/expel
      0.25 ml/min
      Delay (hold): after intake 60 sec, after expel 10 sec
      Cycle: 4×
Repeat it times according to the IgG aliquot number.
Wash away excess ligand with PBS
   Program: 900 μl intake/expel
      0.5 ml/min
      Delay (hold): after intake 30 sec, after expel 30 sec
      Cycle: 4×
Cross-link the antibodies to the column surface with DMS/DMP solution
   Program: 500 μl intake/expel
      0.5 ml/min
      Delay (hold): after intake 30 sec, after expel 30 sec
      Cycle: 4×
   Buffer: 150 mM dimethylpimelimidate (DMP) and 150 mM dimethylsuberimidate (DMS) freshly prepared in ice-cold 0.2 M triethanolamine, pH 8.4.
Repeat this step with fresh DMP/DMS solution.
Wash away the DMS/DMP solution with PBS
   Program: 500 μl intake/expel
      0.5 ml/min
      Delay (hold): after intake 30 sec, after expel 30 sec
      Cycle: 4×
Block any remaining active groups. Transfer the medium to a quenching buffer.
   Program: 500 μl intake/expel
      0.5 ml/min
      Delay (hold): after intake 30 sec, after expel 30 sec
      Cycle: 4×
   Buffer: 1 M ethanolamine, pH 8.0
Repeat this step with fresh ethanolamine solution.
Wash the medium with PBS
   Program: 500 μl intake/expel
      0.5 ml/min
      Delay (hold): after intake 30 sec, after expel 30 sec
      Cycle: 4×
Repeat this step with new PBS.
Optional step: “Empty elution”. Make the elution step (Step 6) to elute the unbound IgG. After wash with PBS.

### 3. Antigen Binding

Should be performed with chilled solutions. CnBr MIA results about ˜600 μl concentrated Eluate at 1.5 μg/μl
MIA eluate is in 0.1 M glycine-HCl, pH 2.7 neutralized with 1 M Tris-HCl, pH 9.0
   Optional: after buffer change is in PBS.
   Final volume: 100 μl
   Total protein to the phynexus tips: 50 ug/tip

MIA eluate: mixed and concentrated peak fractions from Multi Immunoaffinity Column chromatography.
Add the MIA eluate separately to the Phynexus tips
   Program: 150 μl intake/expel
      0.25 ml/min
      Delay (hold): after intake 60 sec, after expel 10 sec
      Cycle: 5×

### 5. Washing

Use a type of detergent to decrease the unspecific binding, like Triton-X100 with cooled buffers.
Wash the column with PBS
   Program: 900 μl intake/expel
      0.5 ml/min
      Delay (hold): after intake 30 sec, after expel 10 sec
      Cycle: 4×
Wash the column with Wash Buffer I
   Buffer: 140 mM NaCl, 50 mM NaH2PO4, pH 6.0, 1% TX-100
   Program: 900 μl intake/expel
      0.5 ml/min
      Delay (hold): after intake 30 sec, after expel 10 sec
      Cycle: 4×
Repeat this step 4 times with new buffer.
Wash the column with Wash Buffer II
   Buffer: 140 mM NaCl, 50 mM NaH2PO4, pH 6.0
   This step is for to remove the residual Tx-100 from the column.
      Program: 900 μl intake/expel
         0.5 ml/min
         Delay (hold): after intake 30 sec, after expel 10 sec
         Cycle: 4×

### 6. Elution:

Elute the bound fraction with 100 μl Elution buffer.
   Buffer: 0.1 M glycine-HCl, pH 2.0
   Program: 100 μl intake/expel
      0.5 ml/min
      Delay (hold): after intake 20 sec, after expel 10 sec
      Cycle: 4×
Neutralize eluted fractions with 20 μl Neutralization Buffer
   Buffer: 1 M Tris-HCl, pH 9.0

### 7. Composition Analysis of Eluted and Flow Through Fractions: Electrophoresis, Comparison (E.G. Automated Image Analysis) in Gel Digestion, Digestion in Liquid Phase, MS.

### 8. Regeneration and Storage of Column

Wash the column with PBS
   Program: 900 μl intake/expel
      0.5 ml/min
      Delay (hold): after intake 30 sec, after expel 30 sec
      Cycle: 4×
Wash and store the column in PBS,
   Program: 900 μl intake/expel
      0.5 ml/min
      Delay (hold): after intake 30 sec, after expel 30 sec
      Cycle: 4×
Phynexus tips should be stored at 4-8° C. in presence of a glycerol

TABLE 1
Output of MS based protein ID (GenBank accession number, gene names, predicted
Mw., confidence level and number of peptides) of material out from boxed areas of the
SDS PAGE gels of eluted samples.

| | | | | |
|---|---|---|---|---|
| Accession # | Name | Confidence | Peptide Found | Biological Process |

20070425IgG1

| | | | | | |
|---|---|---|---|---|---|
| gi\|81175238 | Complement CA-A precursor (Acidic complement CA) | 192771 Da | 99 | 3 | Serum |
| gi\|81175167 | Complement CA-B precursor (Basic complement CA) | 192793 Da | 99 | 3 | Serum |

20070425IgG3A

| | | | | | |
|---|---|---|---|---|---|
| gi\|123508 | Haptoglobin precursor | 45205 Da | 99 | 11 | Serum |
| gi\|123510 | Haptoglobin-related protein precursor | 39008 Da | 99 | 8 | Secreted (potential) |

20070425IgG3B

| | | | | | |
|---|---|---|---|---|---|
| gi\|123508 | Haptoglobin precursor | 45205 Da | 99 | 7 | Serum |
| gi\|123510 | Haptoglobin-related protein precursor | 39008 Da | 99 | 4 | Serum |

20070425IgG5

| | | | | | |
|---|---|---|---|---|---|
| gi\|416733 | CAb-binding protein alpha chain precursor (CAbp) | 67033 Da | 99 | 2 | Serum |

20070425IgG7

| | | | | | |
|---|---|---|---|---|---|
| gi\|399140 | Complement C1q subcomponent subunit 8 precursor | 26459 Da | 99 | 4 | Serum |

TABLE 2
Protein IDs of mAbs 1, 3, 5 and 7.

New IDs:

| | |
|---|---|
| 1. E2 214.11.4 | Complement C4-A precursor |
| 3. E2 84.4 | Haptoglobin-related protein/Haptoglobin |
| 5. E2 223.5 | C4b-binding protein alpha chain precursor |
| 7. E2 235.2 | Complement C1q subcomponent subunit B precursor |

1. A method of protein isolation and/or identification comprising the steps of:
contacting a sample containing proteins with a multi-affinity chromatography support comprising a plurality (e.g., from 3 to 50, from 5 to 40, from 5 to 30, from 5 to 20) of binding reagents;
recovering the material bound to said multi-affinity chromatography support;
separately contacting said material with a plurality of single-affinity chromatography supports, each of said single-affinity chromatography supports comprising a single binding reagent as contained in the multi-affinity chromatography support; and
isolating and/or identifying a protein that binds to a binding reagent in at least one of said single-affinity chromatography supports.

2. The method of claim 1, wherein the sample is a complex sample comprising a plurality of proteins, such as a biological fluid, e.g., plasma, blood, serum, particularly of human origin.

3. The method of claim 2, wherein the sample is pre-treated, e.g., to normalize or reduce the complexity of the sample components.

4. The method of claim 1, wherein the binding reagents are specific for components of a complex biological fluid, such as human plasma.

5. The method of claim 1, wherein the binding reagents are monoclonal antibodies, or derivatives thereof (e.g., Fab fragments, ScFv, etc.) having essentially the same antigen specificity.

6. The method of claim 1, wherein the chromatography supports are columns.

7. The method of claim 1, wherein the sample is human plasma or serum, and the binding reagents are monoclonal antibodies.

8. The method of claim 1, wherein the multiaffinity chromatography support is prepared by loading and crosslinking a plurality of different purified mAbs or mAbs from hybridoma supernatant via their Fc portion onto an appropriate affinity chromatography column, preferably selected from HiTrap Protein G, Protein -L and CNBr activated Sepharose, and allowing the rest of the hybridoma supernatants, or non bound mAbs, to exit the system.

9. The method of claim 1, wherein the multiaffinity chromatography step if performed by loading the appropriate amount of sample on the support; extensive washing to remove any remaining non-specifically bound species, and eluting the bound antigens from the mAb(s) covered affinity surface.

10. The method of claim 1, wherein the single affinity support is prepared by loading and crosslinking each individual mAbs via their Fc portion onto an affinity microcolumn array, preferably selected from Protein G and Protein L pipet tips.

11. The method of claim 1, wherein the single affinity chromatography step if performed by loading an appropriate portion of the product of step 1 onto the individual columns/tips of the microcolumn array and eluting the bound antigens from the individual affinity microcolumn array columns.

12. The method of claim 1, wherein the protein identification step comprises subjecting an eluate of at least one of said single-affinity chromatography supports to an electrophoresis, and isolating a protein band of interest.

13. The method of claim 12, wherein both the eluate and flow through of said at least one single-affinity chromatography support are subjected to electrophoresis, and a protein band that is specific or amplified in the eluate as compared to the flow through is isolated.

14. The method of claim 1, wherein said identification step comprises:
1. Slab gel electrophoresis of the individual flow through and eluate samples from the affinity microcolumn array;
2. Comparative analysis for the identification of mAb specific depleted (flow through) and corresponding enriched (eluate) composition (e.g. stained gel images);
3. After visualization, cut off bands of interest;
4. In gel digestion of cutoff bands, sample preparation, digestion and nano LC-MS analysis to provide protein ID.

15. The method of claim 1, for identifying antigens that are bound by monoclonal antibodies.

16. The method of claim 1, for identifying antigens characteristic of a disease or trait in a mammal, preferably a human.

17. A method of identifying antigens specific for a disease or trait of a mammal, comprising the steps of (i) producing antibodies specific for components of a fluid from said mammal and (ii) identifying antigens recognized by said antibodies using a method of claim 1.
